(19) United States
(12) Patent Application Publication
(10) Pub. No.: US 2009/0214442 A1
(43) Pub. Date: Aug. 27, 2009

(54) Oral Transmucosal Nicotine Dosage Form

(75) Inventors: Vikas Agarwal, Plymouth, MN (US); Brian I. Hague, Salt Lake City, UT (US); Rajendra K. Khankari, Maple Grove, MN (US)

Correspondence Address: Ross J. Oehler;CEPHALON, Inc., 41 MOORES ROAD, PO BOX 4011, FRAZER, PA, 19355, US

(73) Assignees: Cephalon, Inc., Frazer, PA (US); CIMA LABS INC., Eden Prairie, MN (US)

(21) Appl. No.: 12/246,133

(22) Filed: Oct. 6, 2008

Related U.S. Application Data
(63) Continuation-in-part of application No. 11/986,097, filed on Nov. 20, 2007.
(60) Provisional application No. 60/872,177, filed on Dec. 1, 2006.
(60) Provisional application No. 60/872,125, filed on Dec. 1, 2006.

(51) Int. Cl. A61K 9/12; A61K 31/465

(52) U.S. Cl. 424/43; 514/343

(57) ABSTRACT

Described herein are oral transmucosal solid dosage forms useful in treating nicotine addiction or as a nicotine substitute or replacement. By virtue of the formulation in combination with nicotine, the dosage forms transmucosally delivers an effective amount of nicotine to the recipient while permitting the accomplishing of such, and manufacture of such, using a relatively small, convenient and orally comfortable dosage form (e.g., tablet) size.

### RELATED APPLICATION DATA

This application is a continuation in part of and claims the benefit of priority to U.S. application Ser. No. 11/986,097, filed Nov. 20, 2007, which claims priority to U.S. provisional application Nos. 60/872,177 and 60/872,125, both of which were filed on Dec. 1, 2006. The entire contents of all of the referenced applications are hereby incorporated by reference.

### BACKGROUND

A wide variety of nicotine cessation products and therapies are known. Such products include lozenges, gums, transdermal patches, and the like. Lozenges and gums provide oral delivery of nicotine, whereas transdermal patch treatments deliver nicotine through the wearer's skin. These systems are founded on the premise that successful smoking cessation programs require control of the craving episodes associated with nicotine addiction. One example of an oral lozenge-type product is available commercially as COMMIT® (Glaxo-Smithkline, Philadelphia, Pa.). These lozenges are relatively bulky and large in size, and are intended to be swished around within the mouth of the user. Thus, a significant amount of the nicotine can be swallowed, and the delivery of nicotine can be delayed. Further, as with oral gastrointestinal route nicotine treatments, nicotine ingested is subject to first pass metabolism which further reduces systemic delivery of the desired effective amount of active.

Oral transmucosal delivery of nicotine is known. Passive introduction of nicotine to mucosal tissue, such as that introduced by NICORETTE™ gum, can deliver amounts of nicotine transmucosally. One problem, however, is that the administration mechanism or dosage form is heavily commingled with the recipient's saliva, and the active ingredient gets “diluted” within the recipient's oral cavity. Further, the systemic receipt of the active can be significantly delayed.

Delayed delivery of nicotine to a recipient experiencing a nicotine “craving” to rapidly offset the craving can often determine the success or failure of a nicotine cessation product or program. In order to address a craving episode promptly, it is desirable to achieve a front-loaded nicotine delivery to the user's system.

There exists a need in the field of nicotine cessation or replacement therapy and products for an improved oral dosage forms that effectively and rapidly deliver nicotine to a recipient. The present disclosure fulfills this need and has related advantages.

### SUMMARY

Provided herein are oral transmucosal nicotine dosage forms. In one embodiment, the dosage forms utilize effervescence and localized pH adjustment to effectively and rapidly deliver a therapeutically effective amount of nicotine (or nicotine derivative) to a recipient. As further described below, nicotine can be effectively delivered transmucosally using an effervescent solid dosage form intended for static resident placement adjacent the recipient's mucosal tissue. An added benefit of the present disclosure is that because of the enhanced bioavailability of the dosage forms provided herein, smaller tablets can be manufactured to deliver effective amounts of nicotine, thereby permitting more convenient packaging, cost effective manufacturing, and a more comfortable oral administration experience

Provided herein are solid oral transmucosal dosage forms comprising nicotine or nicotine derivative; an effervescent system, and a pH adjusting agent. The dosage form being formulated for static resident placement within a recipient's oral cavity for transmucosal delivery of the nicotine or nicotine derivative across the recipient's oral mucosal tissue. In one embodiment, the dosage form is in the form of a buccal tablet.

As a result of the enhanced transmucosal transport afforded by the dosage forms provided herein, a smaller amount of nicotine in the formulation can effectively deliver a relatively large amount of nicotine to the recipient (Cₘₐₓ) in a relatively short time period (Tₘₐₓ). One benefit associated with certain embodiments of the present disclosure is that by virtue of the combination of ingredients, a given effective nicotine dosage can be achieved with a relatively smaller tablet weight or size because of the achievable earlier bioavailability (e.g., Cₘₐₓ of about 9 ng/ml to about 10 ng/ml as soon as Tₘₐₓ of about 15 minutes to about 50 minutes after placement in the oral cavity in a mammal) afforded by the dosage forms provided herein is comparable to existing commercial products despite the relatively small tablet size (e.g., approximately 5/16″ in one embodiment).

The dose of nicotine or nicotine derivative contained in the dosage forms provided herein can be adjusted to achieve the desired Cₘₐₓ. The compositions formulated for the studies described below were formulated to deliver a Cₘₐₓ of about 9 ng/ml to about 61 ng/ml. It will be understood, however, that the dosage forms can be prepared as described herein which accomplish a variable Cₘₐₓ based on desired effect. For nicotine substitution purposes and smoking cessation purposes, the composition can be formulated to deliver a Cₘₐₓ ranging from about 3 ng/ml to about 70 ng/ml (and a Tₘₐₓ of about 15 minutes to about 60 minutes) or about 7 ng/ml to about 50 ng/ml (and a Tₘₐₓ of about 25 minutes to about 55 minutes). In certain embodiments, the dosage forms provided herein are formulated to provide a Cₘₐₓ ranging from about 7 ng/ml to about 25 ng/ml (and Tₘₐₓ of about 5 minutes to about 50 minutes).

Provided are solid oral transmucosal dosage forms comprising a nicotine or nicotine derivative; an effervescent system; and optionally a pH adjusting substance; wherein the dosage form is formulated for resident placement within a recipient's oral cavity and for transmucosal delivery of the nicotine or nicotine derivative across the recipient's oral mucosal tissue. In certain embodiments, the dosage form is capable of buffering the local pH of the site of administration to a pH of about 6 to about 11, from about 7 to about 10, from about 7 to about 9, or from about 7 to about 8. In certain embodiments, the dosage form is a buccal tablet.

Also provided is a method for treating nicotine addiction in a recipient desiring such treatment, the method comprising: a) providing to the recipient a solid oral dosage form comprising nicotine or nicotine derivative; an effervescent system; and a pH adjusting substance; wherein the dosage form is formulated for resident placement within a recipient's oral cavity and for transmucosal delivery of the nicotine or nicotine derivative across the recipient's oral mucosal tissue; b) positioning the dosage form within the recipient's oral cavity adjacent to oral mucosal tissue; and c) permitting the dosage form to reside in such position for a period of time sufficient to permit at least some of nicotine or nicotine derivative to transport across the oral mucosal tissue. In one embodiment, the method can provide a Cₘₐₓ from about 3 ng/ml to about 70 ng/ml at a Tₘₐₓ of about 10 minutes to about 60 minutes to the recipient.

Also provided is an oral transmucosal nicotine delivery system comprising a solid oral transmucosal dosage form comprising: nicotine or nicotine derivative; an effervescent system; and a pH adjusting substance; the dosage form being formulated for placement within a recipient's oral cavity for transmucosal delivery of nicotine or nicotine derivative across the recipient's oral transmucosal tissue; in combination with a holder; wherein the dosage form is coupled to an end of the holder. In one embodiment, the holder is a hand-held stick.

These and other features and advantages of the present disclosure will become apparent from the following disclosure.

### BRIEF DESCRIPTION OF THE DRAWINGS

The following figures further illustrate the present disclosure, and none are intended to imply a necessary limitation.

FIG. 1 is a chart showing comparative mean plasma concentrations versus time for various solid nicotine dosage form formulations.

FIG. 2 is an illustration of a transmucosal nicotine delivery system with a dosage form on holder, according to one embodiment provided herein.

FIG. 3 demonstrates mean plasma nicotine concentrations versus time for various embodiments herein.

### DETAILED DESCRIPTION

As used herein, the phrase “oral transmucosal,” within the context of drug delivery and absorption, is meant to refer to the pre-peristaltic stage of uptake of the drug via one or more of the mucosal tissue types associated with the oral cavity, e.g., sublingual, buccal, gingival, palatal, esophageal regions of oromucosal tissue. More specifically, what is intended by the phrase is that the primary delivery route of the active ingredient occurs through the mucosal tissue of the oral cavity.

As used herein, the term “about” refers to a range of values from ±10% (i.e., ±1%, ±2%, ±3%, ±4%, ±5%, ±6%, ±7%, ±8%, ±9% and ±10%) of a specified value, and functional equivalents thereof unless otherwise specifically precluded. For example, the phrase “about 50 mg” includes ±10% of 50, or from 45 mg to 55 mg.

As used herein, the term “therapeutically effective amount” is meant to refer to the amount determined to be required to produce the physiological effect intended and associated with the given active ingredient, as measured according to established pharmacokinetic methods and techniques, for the given administration route.

As used herein, the phrase “oral dosage form”, when used in the general sense, includes orally disintegrable/dissolvable tablets, capsules, caplets, gels, creams, films, sprays, and the like. Within the specific context of the instant disclosure, the term “oral dosage form” as it relates to the dosages provided herein refers to the pharmaceutical composition of the instant disclosure as a solid oral dosage form comprising a nicotine or nicotine derivative, accompanied by an excipient formulation which facilitates and enhances oral transmucosal absorption of the active ingredient(s).

As used herein, the term “substantially”, unless otherwise defined, is meant to refer to a specific property, characteristic or variable that meets the stated criteria in such measure that one skilled in the art would understand that the benefit to be achieved, or the condition or property desired, is met.

The compositions provided herein within a general context of being “formulated for resident placement within a recipient's oral cavity for transmucosal delivery of the nicotine or nicotine derivative across the recipient's oral mucosal tissue.” This phrase, and like phrases made herein, are meant to indicate that by virtue of the collective combination of ingredients, their individual and combined functionalities, and the techniques used to prepare the dosage form, provide a dosage form that affords delivery of the active ingredient (nicotine) across the recipient's mucosal tissue when placed adjacent thereto for a period of time sufficient to permit such transport.

Compositions prepared as provided herein contain nicotine or a nicotine derivative as an active pharmaceutical ingredient. Suitable nicotine derivatives that can be used include pharmaceutically acceptable resin complexes and pharmaceutically acceptable salts of nicotine. Suitable nicotine derivatives include, but are not limited to, nicotine polacrilex and nicotine bitartrate. For therapeutic effect for smoking cessation purposes (i.e., delivery of nicotine in an amount sufficient to address the craving episode), the absorbed amount needed can vary.

For compositions prepared as described herein, dose amounts of nicotine that can be used can range from about 0.5 mg to about 10.0 mg from about 0.5 mg to about 9.0 mg, 0.5 mg to about 8 mg, 0.5 mg to about 7.0 mg, 0.5 mg to about 6.0 mg, or 0.5 mg to about 5.0 mg but are variable based on the desired therapy, results or effect. In certain embodiments, the dose of nicotine provided in the compositions described herein is about 2.0 mg. Within a smoking cessation context, dosage forms prepared according to the this disclosure can be administered with a frequency sufficient to achieve a total daily dosage amount of up to about 60 mg/day. The total daily dosage of nicotine or nicotine derivative desired will vary according to the individual's specific therapeutic, cessation or substitution needs, preferences or requirements.

The nicotine compositions prepared as described herein comprise an effervescent system and a pH adjusting substance. A variety of effervescent systems can be used in the dosage forms provided herein. For example, certain types of effervescent systems are described in U.S. Pat. No. 5,178,878 and U.S. Pat. No. 5,503,846 and can be used in the dosage forms described herein; the entire texts of these patents are incorporated herein by reference.

The term “effervescent system” as used herein is meant to encompass one or more food grade compounds, that upon reaction, e.g., with one and another, with another component of the dosage forms described herein, and/or with a naturally occurring component(s) of saliva, under physiological conditions, generates a gas. Gases may include, but are not limited to oxygen, nitrogen, or carbon dioxide. One example of an effervescent system comprises a food grade acid or salt thereof, and a food grade bicarbonate or carbonate base, that upon reaction with the food grade acid generates carbon dioxide. The food grade acid may contain one or more acidic protons, which are of a suitable pKa to react with the carbonate or bicarbonate base. Food grade acids may have one or more acid exchangeable protons (e.g., mono-, di-, tri-, and tetra-protic acids). Food grade acids include organic acids, inorganic acids, and organic acid anhydrides, and salts thereof. The pKa of acid exchangeable proton(s) of the food grade acid can range from about 1 to about 11, from about 2 to about 10, from about 2 to about 9, from about 2 to about 8, from about 2 to about 7, from about 2 to about 6, and from about 3 to about 7.

Where the effervescent system includes two or more mutually reactive components, such as a food grade acid and a food grade base, when the components are not present in substantially equal stoichiometric molar amounts (e.g., where there is a stoichiometric excess of one component assuming complete reaction), the effervescent system described herein encompasses the stoichiometric amount of each component present. For example, if the acid used is diprotic, then either substantially twice the molar amount of a mono-reactive base, or a substantially equal molar amount of a di-reactive base is encompassed by the effervescent system. In certain embodiments, the amount of either the food grade acid or food grade base source may exceed the amount of the other component. The excess agent may be used to adjust the localized pH.

Effervescent systems include those that are water- or saliva-activated materials usually kept in anhydrous state with little or no absorbed moisture, or in a stable hydrated form.

Acids for use in the effervescent system include food grade organic and inorganic acids, acid anhydrides and acid salts. Acids include, but are not limited to, citric acid, tartaric acid, benzoic acid, malic acid, citric acid, lactic acid, gluconic acid, fumaric acid, adipic acid, ascorbic acid and succinic acid, phosphoric acid, and acid anhydrides or salts thereof. Acid salts may include dihydrogen phosphate, dihydrogen pyrophosphate, and hydrogen phosphate, acid sulfite salts and acid citrate salts and monohydrogen sulfate. In certain embodiments, citric acid is used.

Bases that can be used include, but are not limited to, carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate and amorphous calcium carbonate.

Sodium carbonate, potassium carbonate, calcium carbonate, magnesium carbonate and the like can also be used, e.g., as part of the effervescent system, but can also or separately be used as a pH adjusting substance. In some embodiments, for example, if sodium carbonate, potassium carbonate, magnesium carbonate and the like are used as part of the effervescent system but are present in stoichiometric molar excess relative to the acid present, the excess can be a pH adjusting substance. In other embodiments, an effervescent system includes an acid and bicarbonate in about stoichiometric amounts, and a carbonate can be used as a pH adjusting substance.

In certain instances, where nicotine is present as an acid salt or complexed to an acidic resin, the effervescent system can be a single agent comprising a carbonate or bicarbonate salt.

The amount of effervescent system component useful in accordance with the present disclosure is an effective amount and is determined based on properties other than those which would be necessary to achieve disintegration of a tablet in the mouth. Instead, effervescence is used in the dosage forms provided as a basis for enhancing transmission of the active ingredient across mucosal membranes via buccal, sublingual or gingival administration in the oral cavity. Accordingly, the amount of effervescent system should range between about 5 to about 85 percent, between about 15 and 60 percent, about 30 and 45 percent, or about 35 and 40 percent, based on total formulation weight. Of course, the relative proportion of acid and base will depend upon the specific ingredients, e.g., whether the acid is mono-, di- or tri-protic, relative molecular weights, etc.

Various pH adjusting substances can be used, e.g., to provide permeation enhancement of the active ingredient. The selection of the appropriate pH adjusting substance will depend on the drug to be administered and, in particular, to the pH at which the drug is ionized or unionized, and whether the ionized form or unionized form facilitates transmission across the mucosa.

In one embodiment, the pH adjusting substance is any substance that is capable of adjusting the localized pH to promote transport across the mucosa in amounts which will result in a pH generally ranging from about 3 to about 10, from about 4 to about 10, from about 5 to about 10, from about 6 to about 10, or from about 7 to about 10. The pH is the “localized pH” at the microenvironment at the surface contact area of the oral mucosa and the dosage form (or portions of it as it disintegrates and/or dissolves) once placed in the mouth of the recipient.

In general, the localized pH can be determined by initially characterizing the dynamic pH changes displayed by the tablets using in vitro pH measurement. The method consists of using 0.5-10 ml phosphate buffered saline in an appropriately sized test tube or other similar vessel. One liter volume of buffered saline solution can be prepared by dissolving 9.0 g sodium chloride, 0.6 g sodium phosphate monobasic monohydrate and 0.78 g of sodium phosphate dibasic (anhydrous) in about 1000 ml of deionized water, and adjusting the pH to 7.0±0.05 at room temperature by adding 1 N sodium hydroxide with stirring. The adjustment should require about 0.5 ml. The amount of media used depends on the tablet size and dosage. For example, a volume of 2 ml can be used for a tablet weighing 200 mg. Immediately upon contact with the media, the pH profile of the solution is monitored as a function of time, using a micro-combination pH electrode.

In certain embodiments, the materials which can be used as pH adjusting substances in accordance with the present disclosure include carbonate, bicarbonate, phosphate, hydrogen phosphate and dihydrogen phosphate. Suitable carbonates include sodium carbonate, potassium carbonate, magnesium carbonate, or calcium carbonate. Suitable phosphates include calcium phosphate or sodium phosphate. In certain embodiments, the pH adjusting substance is sodium carbonate.

The amount of pH adjusting substance can vary with the type of pH adjusting substance used, amount of excess acid or base, if any, from the effervescent system, the nature of remaining ingredients, and the active ingredient. The amount of pH adjusting substance can vary from about 0.5 to about 25 percent, from about 2 to about 20 percent, from about 5 to about 15 percent, or from about 7 and about 12 percent by weight of the total formulation weight.

In one embodiment, a solid oral transmucosal dosage form comprises:
a) nicotine or a nicotine derivative or a pharmaceutically acceptable salt thereof,
b) an effervescent system; and
c) a pH adjusting substance;
   the dosage form being formulated for resident placement within a recipient's oral cavity for transmucosal delivery of the nicotine or nicotine derivative across the recipient's oral mucosal tissue.

Certain embodiments relate to the aforementioned dosage form and attendant limitations, wherein the pH adjusting substance is present in a amount sufficient to provide a localized pH in the range of about 7 to about 9. In certain embodiments the pH adjusting substance is present in a amount sufficient to provide a localized pH in the range of about 3 to about 10, about 4 to about 10, about 5 to about 10, about 6 to about 10, about 7 to about 10, or about 7 to about 8.

Certain embodiments relate to any one of the aforementioned dosage forms and attendant limitations, wherein the effervescent system comprises an acid and a base.

Certain embodiments relate to any one of the aforementioned dosage forms and attendant limitations, wherein the base and the pH adjusting substance are the same compound.

Certain embodiments relate to any one of the aforementioned dosage forms and attendant limitations, wherein the base is an alkali metal carbonate salt, an alkali metal bicarbonate salt, an alkaline earth carbonate, or an alkaline earth bicarbonate.

Certain embodiments relate to any one of the aforementioned dosage forms and attendant limitations, wherein the acid is citric acid.

Certain embodiments relate to any one of the aforementioned dosage forms and attendant limitations, wherein the effervescent system is present in amount of about 5% to about 95% by weight of the total dosage form. In certain embodiments the effervescent system is present in amount of about 5% to about 95%, about 5% to about 85%, about 5% to about 75%, about 5% to about 65%, about 15% to about 65%, about 25% to about 65%, or about 25% to about 50% by weight of the total dosage form.

Certain embodiments relate to any one of the aforementioned dosage forms and attendant limitations, wherein the effervescent system comprises citric acid and an alkali or alkaline earth bicarbonate salt.

Certain embodiments relate to any one of the aforementioned dosage forms and attendant limitations, wherein the effervescent system comprises citric acid or tartaric acid and an alkali or alkaline earth bicarbonate salt and the pH adjusting agent is an alkali or alkaline earth carbonate salt.

Certain embodiments relate to any one of the aforementioned dosage forms and attendant limitations, wherein the effervescent system comprises citric acid or tartaric acid and an alkali or alkaline earth bicarbonate salt; and the pH adjusting agent is an alkali or alkaline earth carbonate salt; wherein the effervescent system is present in amount of about 25% to about 50% by weight of the total dosage form

Certain embodiments relate to any one of the aforementioned dosage forms and attendant limitations, wherein the dosage form composition is in the form of a 200 mg total weight oral buccal transmucosal tablet containing nicotine derivative from about 0.5 mg to about 4.0 mg, the tablet having a diameter of about 5/16 inch.

Certain embodiments relate to any one of the aforementioned dosage forms and attendant limitations, wherein the nicotine derivative is selected from the group consisting of nicotine polacrilex and nicotine bitartrate.

Certain embodiments relate to any one of the aforementioned dosage forms and attendant limitations, wherein the dosage form upon administration achieves a Cₘₐₓ to dose ratio ranging from about 1 to about 12 picogram/mL/microgram. For example, the dosage form can achieve a Cₘₐₓ to dose ratio of from about 2 to about 10; from about 2 to about 12; from about 4 to about 10; from about 4 to about 12; from about 6 to about 10; from about 6 to about 12; from about 8 to about 10; or from about 8 to about 12 picrogram/mL/microgram.

Certain embodiments relate to any one of the aforementioned dosage forms and attendant limitations, wherein the dosage form upon administration achieves a Cₘₐₓ to dose ratio ranging from about 3 to about 12 picogram/mL/microgram.

Certain embodiments relate to any one of the aforementioned dosage forms and attendant limitations, wherein the dosage form upon administration achieves a Cₘₐₓ to dose ratio ranging from about 3 to about 6.3 picogram/mL/microgram.

Certain embodiments relate to any one of the aforementioned dosage forms and attendant limitations, wherein the dosage form upon administration achieves a Cₘₐₓ to dose ratio ranging from about 3.7 to about 6.3 picogram/mL/microgram.

Also provided is a solid oral transmucosal dosage form comprising the following ingredients:
a) nicotine or nicotine derivative or a pharmaceutically acceptable salts thereof, and
b) an effervescent system consisting essentially of an acid and an alkali metal or alkaline metal bicarbonate or carbonate salt;
   the dosage form being formulated for resident placement within a recipient's oral cavity for transmucosal delivery of the nicotine or nicotine derivative across the recipient's oral mucosal tissue; wherein the bicarbonate or carbonate salt is present in stoichiometric excess relative to the acid and the amount is sufficient to provide a localized pH in the range of about 4 to about 10.

In certain embodiments the pH adjusting substance is present in a amount sufficient to provide a localized pH in the range of about 3 to about 10, about 4 to about 10, about 5 to about 10, about 6 to about 10, about 7 to about 10, or about 7 to about 8.

Certain embodiments relate to the aforementioned dosage form and attendant limitations, wherein the effervescent is present in amount of about 25% to about 50% by weight of the total dosage form.

Certain embodiments relate to any one of the aforementioned dosage forms and attendant limitations, wherein the dosage form upon administration achieves a Cₘₐₓ to dose ratio ranging from about 1 to about 12 picogram/mL/microgram.

Certain embodiments relate to any one of the aforementioned dosage forms and attendant limitations, wherein the dosage form upon administration achieves a Cₘₐₓ to dose ratio ranging from about 3 to about 12 picogram/mL/microgram.

Certain embodiments relate to any one of the aforementioned dosage forms and attendant limitations, wherein the dosage form upon administration achieves a Cₘₐₓ to dose ratio ranging from about 3 to about 6.3 picogram/mL/microgram.

Certain embodiments relate to any one of the aforementioned dosage forms and attendant limitations, wherein the dosage form upon administration achieves a Cₘₐₓ to dose ratio ranging from about 3.7 to about 6.3 picogram/mL/microgram.

Certain embodiments relate to any one of the aforementioned dosage forms and attendant limitations, wherein the dosage form upon administration has an AUC_{0-inf} of about 15 to about 40, 18 to about 33, or about 20 to about 30 nghr/mL.

Certain embodiments relate to any one of the aforementioned dosage forms and attendant limitations, wherein the dosage form upon administration achieves a Tₘₐₓ in the range of about 5 to about 90, about 15 to about 90, about 25 to about 90, about 35 to about 80, about 35 to about 70, about 35 to about 60, or about 35 to about 50 minutes.

Another embodiment relates to any of the aforementioned dosage forms and attendant limitations, wherein the dosage form upon administration achieves a Tₘₐₓ of about 2 to about 60, about 5 to about 50, about 10 to about 40, about 10 to about 30 or about 10 to about 25 minutes.

Another embodiment relates to any of the aforementioned dosage forms and attendant limitations, wherein the dosage upon administration achieves a Tₘₐₓ of about 2 to about 60, about 5 to about 50, about 10 to about 40, about 10 to about 30 or about 10 to about 25 minutes.

Another embodiment relates to any of the aforementioned dosage forms and attendant limitations, wherein the dosage form upon administration achieves a Tₘₐₓ in less than about 60, less than about 50 minutes, less than about 45 minutes, less than about 40, less than about 35 minutes, less than about 30 minutes, less than about 25 minutes, or less than about 20 minutes.

Also provided is a method of treating nicotine addiction in a recipient desiring such treatment, the method comprising:
a) providing to the recipient a solid oral transmucosal dosage form comprising:
   i) nicotine or nicotine derivative or a pharmaceutically acceptable salts thereof,
   ii) an effervescent system; and
   iii) a pH adjusting substance;
the dosage form being formulated for resident placement within a recipient's oral cavity for transmucosal delivery of the nicotine or nicotine derivative across the recipient's oral mucosal tissue;
b) positioning the dosage form within the recipient's oral cavity adjacent to oral mucosal tissue; and
c) permitting the dosage form to reside in such position for a period of time sufficient to permit the nicotine or nicotine derivative to transport across the oral mucosal tissue;
   wherein step c) and the dosage form achieves a Cₘₐₓ to dose ratio in a range of from about 1 to about 12 picogram/mL/microgram.

Certain embodiments relate to the aforementioned dosage form and attendant limitations, wherein the dosage form upon administration achieves a Cₘₐₓ to dose ratio ranging from about 3 to about 12 picogram/mL/microgram.

Certain embodiments relate to any one of the aforementioned dosage form and attendant limitations, wherein the dosage form upon administration achieves a Cₘₐₓ to dose ratio ranging from about 3 to about 6.3 picogram/mL/microgram.

Certain embodiments relate to any one of the aforementioned methods and attendant limitations, wherein the method has an AUC_{0-inf} of about 15 to about 40, 18 to about 33, or about 20 to about 30 nghr/mL.

Certain embodiments relate to any the aforementioned methods and attendant limitations, wherein the method achieves a Tₘₐₓ, in the range of about 5 to about 90, about 15 to about 90, about 25 to about 90, about 35 to about 80, about 35 to about 70, about 35 to about 60, or about 35 to about 50 minutes.

Another embodiment relates to aforementioned methods and attendant limitations, wherein the method achieves a Tₘₐₓ, of about 2 to about 60, about 5 to about 50, about 10 to about 40, about 10 to about 30 or about 10 to about 25 minutes.

Another embodiment relates to any of the aforementioned methods and attendant limitations, wherein the method achieves a Tₘₐₓ of about 2 to about 60, about 5 to about 50, about 110 to about 40, about 110 to about 30 or about 110 to about 25 minutes.

Another embodiment relates to any of the aforementioned method and attendant limitations, wherein the method achieves a Tₘₐₓ in less than about 60, less than about 50 minutes, less than about 45 minutes, less than about 40, less than about 35 minutes, less than about 30 minutes, less than about 25 minutes, or less than about 20 minutes.

Also provided is an oral transmucosal nicotine delivery system, the system comprising:
a) a solid oral transmucosal dosage form comprising a composition having the following ingredients:
   i) nicotine or nicotine derivative or a pharmaceutically acceptable salt thereof,
   ii) an effervescent system; and
   iii) a pH adjusting substance;
the dosage form being formulated for placement within a recipient's oral cavity for transmucosal delivery of the nicotine or nicotine derivative across the recipient's oral mucosal tissue; in combination with
b) a holder;
   the dosage form being coupled to an end of the holder.

Certain embodiment relate to the aforementioned delivery system and attendant limitations, wherein the holder is a hand-held stick.

Certain embodiment relate to any one of the aforementioned delivery systems and attendant limitations, wherein the delivery system provides a localized pH in the range of about 7 to about 9.

In certain embodiments the pH adjusting substance is present in a amount sufficient to provide a localized pH in the range of about 3 to about 10, about 4 to about 10, about 5 to about 10, about 6 to about 10, about 7 to about 10, or about 7 to about 8.

Certain embodiment relate to any one of the aforementioned delivery systems and attendant limitations, wherein the effervescent system is present in an amount of about 25% to about 50% by weight of the total dosage form.

Certain embodiments relate to any one of the aforementioned delivery systems and attendant limitations, wherein the delivery systems achieves a Cₘₐₓ to dose ratio ranging from about 1 to about 12 picogram/mL/microgram.

Certain embodiments relate to any one of the aforementioned delivery systems and attendant limitations, wherein the delivery systems achieves a Cₘₐₓ to dose ratio ranging from about 3 to about 12 picogram/mL/microgram.

Certain embodiments relate to any one of the aforementioned delivery systems and attendant limitations, wherein the delivery systems achieves a Cₘₐₓ to dose ratio ranging from about 3 to about 6.3 picogram/mL/microgram.

Certain embodiments relate to any one of the aforementioned delivery systems and attendant limitations, wherein the delivery systems achieves a Cₘₐₓ to dose ratio ranging from about 3.7 to about 6.3 picogram/mL/microgram.

Certain embodiments relate to any one of the aforementioned delivery systems and attendant limitations, wherein the delivery systems has an AUC0-inf of about 15 to about 40, 18 to about 33, or about 20 to about 30 nghr/mL.

Certain embodiments relate to any one of the aforementioned delivery systems and attendant limitations, wherein the delivery systems achieves a Tₘₐₓ in the range of about 5 to about 90, about 15 to about 90, about 25 to about 90, about 35 to about 80, about 35 to about 70, about 35 to about 60, or about 35 to about 50 minutes.

Another embodiment relates to any of the aforementioned delivery systems and attendant limitations, wherein the delivery systems achieves a Tₘₐₓ of about 2 to about 60, about 5 to about 50, about 10 to about 40, about 10 to about 30 or about 10 to about 25 minutes.

Another embodiment relates to any of the aforementioned delivery systems and attendant limitations, wherein the delivery systems achieves a Tₘₐₓ in less than about 60, less than about 50 minutes, less than about 45 minutes, less than about 40, less than about 35 minutes, less than about 30 minutes, less than about 25 minutes, or less than about 20 minutes.

Certain embodiment relate to any one of the aforementioned delivery systems and attendant limitations, wherein the holder and dosage form are constructed for reversible coupling to one another.

Also provided is a method of nicotine substitution comprising:
a) providing a dosage form to a recipient desiring the substitution, the dosage form being a solid oral transmucosal dosage form comprising:
   i) nicotine or nicotine derivative or a pharmaceutically acceptable salt thereof,
   ii) an effervescent system; and
   iii) a pH adjusting substance;
the dosage form being formulated for placement within a recipient's oral cavity for transmucosal delivery of the nicotine or nicotine derivative across the recipient's oral mucosal tissue;
b) placing the dosage form within the recipient's oral cavity adjacent to recipient's mucosal tissue; and
c) permitting the dosage form to reside adjacent the mucosal tissue for a period of time sufficient to deliver nicotine across the mucosal tissue.

Certain embodiments relate to the aforementioned method and attendant limitations, where the method provides a localized pH in the range of about 7 to about 9.

In certain embodiments the pH adjusting substance is present in a amount sufficient to provide a localized pH in the range of about 3 to about 10, about 4 to about 10, about 5 to about 10, about 6 to about 10, about 7 to about 10, or about 7 to about 8.

Certain embodiments relate to any one of the aforementioned methods and attendant limitations, wherein the effervescent system is present in an amount of about 25% to about 50% by weight of the total dosage form.

In certain embodiments, the dosage from provided herein comprises a filler, disintegrant, or lubricant, and combinations thereof. Any filler or any amount of a filler can be used as long as the resulting dosage forms achieve the results described herein. In certain embodiments the fillers are sugar and sugar alcohols, and these may include non-direct compression and direct compression fillers. Non-direct compression fillers generally, at least when formulated, have flow and/or compression characteristics which make them impractical for use in high speed tableting process without augmentation or adjustment. For example, a formulation may not flow sufficiently well and therefore, a glidant such as silicon dioxide may need to be added.

Direct compression fillers, by contrast, do not require similar allowances. They generally have compressibility and flowability characteristics which allow them to be used directly. It is noted that, depending upon the method by which the formulations are made, non-direct compression fillers may be imparted with the properties of direct compression fillers. The reverse is also true. As a general matter, non-direct compression fillers tend to have relatively smaller particle size when compared to direct compression fillers. However, certain fillers such as spray dried mannitol have relatively smaller particle sizes and yet are often directly compressible, depending on how they are further processed. There are also relatively large non-direct compression fillers as well.

Suitable fillers include, but are not limited to, mannitol, lactose, sorbitol, dextrose, sucrose, xylitol and glucose, to the extent that they can provide the results described herein. In certain instance the filler is spray dried mannitol. The amount of filler used can range from about 10 to about 80 percent, from about 25 to about 80 percent, or from about 25 to about 60 percent by weight of the formulation.

Disintegrants can also be used in the dosage forms provided herein. Disintegrants can permit dosage reduction and/or increase the ratio of Cₘₐₓ and dose. Disintegrants can include binders that also have disintegrant properties. Suitable disintegrants include, but are not limited to, microcrystalline cellulose, cross-linked polyvinyl pyrrolidone (PVP-XL), sodium starch glycolate, croscarmellose sodium, cross-linked hydroxypropyl cellulose, and the like. Selection of the disintegrant can depend upon whether or not, within a given system, the results described can be obtained with its use. In certain instances, the disintegrant is a starch glycolate or a sodium starch glycolate.

The amount of disintegrant can vary according to factors such as dosage form size, nature and amount of other ingredients, and the like. Generally, the amount of disintegrant can range from about 0.25% to about 20% by weight of the final formulation, between about 0.5% and about 15% w/w, between about 0.5% and about 10% w/w, and between about 1% and about 8% by weight—based on the weight of the finished formulation.

In certain embodiments the oral dosage forms comprise a tableting or ejection lubricant. Suitable lubricants include, but are not limited to, magnesium stearate, stearic acid, calcium stearate, and combinations thereof. In certain instances the lubricant is magnesium stearate. The amount of lubricant may be less than 1% or 0.5% of the formulation by weight. In the case of magnesium stearate, the amount can be greater than about 1.0% provided the amount does not adversely affect the desired properties of the resulting dosage form. greater than 1.5% or between about 1.5% and about 3%. When magnesium stearate is used, the amount can be about 2% by weight.

In certain embodiments, additional excipients can be included in the dosage forms provided herein. Additional excipients include, but are not limited to, binders, sweeteners, coloring agents, flavoring agents, glidants, lubricants, disintegrants, preservatives, and the like.

The dosage forms provided herein can be prepared as a solid oral transmucosal dosage form, e.g., tablet. Effervescent tablets prepared in accordance with the instant disclosure can be relatively robust or soft. For example, tablets containing the dosage forms provided herein can generally be prepared according to the manufacturing methods described in U.S. Pat. No. 5,178,878, the text of which is incorporated herein by reference. When prepared according to this technique, the dosage form can have a hardness of less than about 15 Newtons, but the active ingredient need not necessarily be coated with a protective material. When soft friable tablets are produced, they can be advantageously packaged in blister packs such as those described in U.S. Pat. No. 6,155,423. Alternatively, robust dosage forms with a hardness of greater than about 15 Newtons can be manufactured according to the process described in U.S. Pat. No. 6,024,981. Further, the degree of state of powder, e.g., reproducibility and/or consistency of particle size, can affect results.

### EXAMPLES

### Example 1

### Preparation of Packaged Oral Transmucosal Dosage Form (Tablet) Containing 2 mg Nicotine from Nicotine Polacrilex

A 200 mg solid oral transmucosal tablet was prepared having a nicotine polacrilex potency (15%) effective to deliver 2 mg dose active nicotine. Nicotine polacrilex, mannitol (spray-dried), sodium bicarbonate, citric acid, sodium carbonate and sodium starch glycolate were sieved and blended in a mixer for a predetermined period of time (about 30 minutes). After this mixture was prepared, magnesium stearate was then added to the mixture and blended for about 5 minutes. The resultant mixture was then discharged and compressed on a rotary tablet press thereby forming tablets to defined and predetermined weight (200 mg) and hardness (10 N). The tablets were then sorted and packaged into aluminum-aluminum blister packs. The blending, tableting and blister packing operations were all undertaken in humidity controlled environmental conditions of less than 25 grains of moisture per pound dry air.

The resulting tablet contained the following formulation:

TABLE 1
2 mg Nicotine (from Nicotine Polacrilex) Tablet

| Ingredient: | mg/tablet | | % w/w |
|---|---|---|---|
| Nicotine polacrilex (15%) | 13.33 | | 6.67 |
| Mannitol (spray-dried) | 84.67 | | 42.33 |
| Sodium bicarbonate | 42.00 | | 21.00 |
| Citric acid | 30.00 | | 15.00 |
| Sodium carbonate | 20.00 | | 10.00 |
| Sodium starch glycolate | 6.00 | | 3.00 |
| Magnesium stearate | 4.00 | | 2.00 |
| Total: | 200.00 | mg | 100.0% |

* Nicotine polacrilex is based on 15% potency and a 2 mg dose of nicotine.

### Example 2

### Preparation of Oral Transmucosal Dosage Form (Tablet) Containing 2 mg Nicotine from Nicotine Bitartrate

Using a procedure similar to that described above in Example 3, a 200 mg solid oral transmucosal tablet containing nicotine bitartrate as the active nicotine source was prepared.

The formulation appears in the following table:

TABLE 2
2 mg Nicotine (from Nicotine Bitartrate) Tablet

| Ingredient: | mg/tablet | | % w/w |
|---|---|---|---|
| Nicotine bitartrate dihydrate (34%)* | 6.15 | | 3.08 |
| Mannitol (spray-dried) | 91.85 | | 45.92 |
| Sodium bicarbonate | 42.00 | | 21.00 |
| Citric acid | 30.00 | | 15.00 |
| Sodium carbonate | 20.00 | | 10.00 |
| Sodium starch glycolate | 6.00 | | 3.00 |
| Magnesium stearate | 4.00 | | 2.00 |
| Total: | 200.00 | mg | 100.0% |

*Nicotine bitartrate dihydrate is based on 34% potency and a 2 mg dose of nicotine.

### Example 3

### Comparative 2 mg Nicotine (from Nicotine Polacrilex) Formulation

Using a process similar to that described above in Example 1, a 200 mg nicotine tablet formulation was prepared containing no effervescent system and pH adjusting substance ingredients as described herein. The filler ingredient, mannitol, was used to replace the effervescent system and pH adjusting ingredient amounts in the nicotine polacrilex formulation of Example 1.

The resulting formulation is set forth in the following table:

TABLE 3
Comparative 2 mg nicotine (from nicotine polacrilex) Formulation

| Ingredient: | mg/tablet | % w/w |
|---|---|---|
| Nicotine polacrilex (15%)* | 13.33 | 6.67 |
| Mannitol (spray-dried) | 176.67 | 88.33 |
| Sodium starch glycolate | 6.00 | 3.00 |
| Magnesium stearate | 4.00 | 2.00 |
| Total: | 200.00 mg | 100.0% |

*Nicotine polacrilex is based on 15% potency and a 2 mg dose.

### Example 4

### Comparative In Vivo Bioavailability Data

Commercial product formulation COMMIT® Lozenge (available from Glaxo Smithkline Beecham), an oral 2 mg nicotine (from nicotine polacrilex) dosage form, was obtained and used in a comparative experiment. The purpose of the experiment was to evaluate bioavailability or PK parameters associated with four formulations (formulations of Table 1 and Table 2, comparator formulation Table 3 and the commercial product COMMIT®). The 2 mg nicotine COMMIT® lozenge used in the comparison had a lozenge weight of 1225 mg. For purposes of the experiment, the COMMIT® lozenge was placed adjacent the mucosa for static positioning to “mimic” a static buccal transmucosal-type dosage form, despite the instructions associated with the product which instruct swishing around within the oral cavity.

Alongside the solid dosage forms used in the experiment, an intravenously-administered solution was also prepared and used in the experiment to use as the basis for calculating theoretical absolute bioavailability of the solid dosage forms. A 5 ml of 1 mg/ml nicotine solution was prepared by dissolving 15.36 mg nicotine bitartrate dihydrate in water added until a total amount of 5 ml was reached. The solution was prepared based on the nicotine bitartrate dihydrate nicotine base:salt ratio of 3.07. Next, 15.36 mg nicotine bitartate dihydrate was weighed into a tared sterile 5 ml vial, into which was added 5 ml sterile water for injection (SWFI). The solution was aspirated into a 5 ml syringe. Onto the syringe tip was placed a 0.2 micron filter, and a 18 gauge needle was placed onto the filter and the solution transferred through the filter/needle assembly into an empty sterile 5 ml vial. The vial was dated to expire within 24 hours.

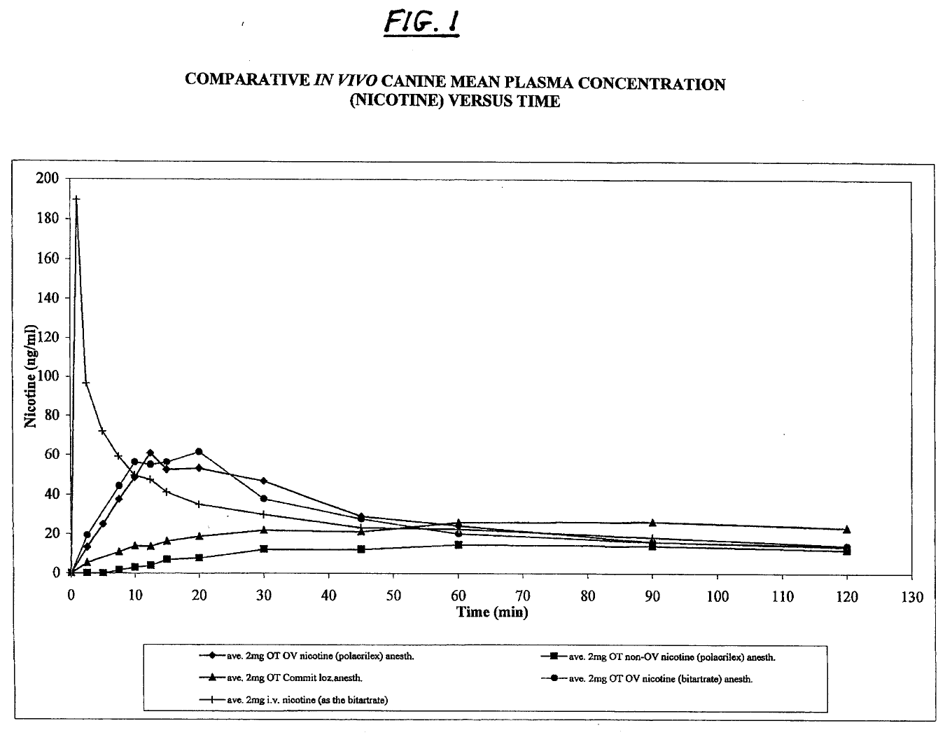

In the in vivo experiment, the i.v. solution was administered to average 2 mg nicotine bitartrate administration at a rate of 1 ml/min for a period of 2 minutes, which corresponded to the highest oral transmucosal dose tested in solid form. Samples were drawn at zero time and predetermined time intervals set forth in FIG. 1 (see 2 mg i.v. nicotine key). After being drawn, the samples were left to stand for 10 minutes and then centrifuged to provide the serum samples for analysis.

For the bucally administered dosage forms, a 5/16 inch diameter dosage unit was placed in the lower buccal cavity of the canine subject opposite to the side of the mouth that was resting on the surgical table. Then, 100 to 200 μl saliva substitute (sodium chloride/sodium phosphate solution adjusted to pH 7.0 using sodium hydroxide) was instilled at t=0 and every 2.5 minutes until the dissolution of the dosage unit was achieved. The subject's mouth was kept open but not stretched with jaw clamp to avoid stress to the masseter muscle. The mouth was washed and wiped before the experiment began and unclamped at 15 minutes after start time. A zero time sample was drawn before placement of the dosage unit in the buccal cavity, followed by arterial samples of appropriate volume drawn at predetermined time intervals (see FIG. 1). The samples are left to stand 10 minutes before centrifuging and serum analysis. In both the solid dosage unit and intravenous testing samples, a dosage averaging 2 mg nicotine was administered.

Each canine subject was restricted to fluids for 12 hours prior to the study and sedated with propofol before intubation. The i.v. line was inserted into the cephalic vein and followed by Normal Saline infusion at approximately 15 ml/kg (480 ml/hr) for 1 hour, then 5 ml/kg (160 ml/hr) for the remaining time. After i.v. line insertion, the subject is then connected to a closed circuit delivering 2% isoflurane. Alternatively, for conscious sedation subjects, alternatively medetomide HCl was administered. An arterial line was inserted in the femoral artery for collection of the arterial blood samples. For conscious sedation, serum samples were obtained via the cephalic line to avoid discomfort and stress on the subject. Sample volumes were recorded.

After centrifugation and serum analysis, the serum concentrations and bioavailability parameters were calculated. The bioavailability data is set forth in the following table and also plotted in FIG. 1.

TABLE 4
Comparative in vivo Canine Bioavailability Data

| | Ex. 1, Table 1 | Ex. 3, Table 3 | Commercial | Ex. 2, Table 2 | I.V. |
|---|---|---|---|---|---|
| Measurement: | 2 mg OT | 2 mg OT | 2 mg “OT” | 2 mg OT | 2 mg i.v. |
| | OV | non-OV | (commercial | OV | nicotine |
| | Nicotine | nicotine | lozenge) | Nicotine | (bitartrate) |
| | (polacrilex) | (polacrilex) | | (bitartrate) | |
| Cₘₐₓ (ave. | 61.33 | 15.67 | 28.33 | 64.67 | 189.61 |
| ng/ml) | | | | | |
| Tₘₐₓ (ave. | 13.33 | 55.00 | 80.00 | 17.50 | 1.00 |
| ng/ml) | | | | | |
| AUC₀₆₁₂₀ | 3085 | 1377 | 2652 | 3228 | 3424.21 |
| Bioavail. | 92.77 ± 25.93 | 42.92 ± 23.33 | 83.35 ± 12.50 | 101.58 ± 9.98 | — |

n = 3
OT = oral transmucosal
OV = formulated with effervescent system and pH adjusting substance.
Non-OV = formulated without effervescent system and pH adjusting substance.

The results were plotted and shown in FIG. 1. As can be seen from the above data, the tablet formulations containing an effervescent system and pH adjusting agent (the two formulations of Example 1 Table 1 and Example 2 Table 2 appearing as “2 mg OV nicotine (polacrilex)” and “2 mg OV nicotine (bitartrate)” respectively) clearly show a substantially higher Cₘₐₓ and substantially shorter Tₘₐₓ as compared to the formulation of Example 3 Table 3 (appearing as “non-OV nicotine”) and comparator product COMMIT®. The oral transmucosal dosage forms containing the effervescent system and pH adjusting ingredients exhibited faster onset action in terms of Cₘₐₓ and Tₘₐₓ bioavailability and pharmacokinetics as compared to even the comparator formulation absent the effervescent system and pH adjusting ingredients.

### Example 5

### Large Scale Preparation of 200 mg Tablet Containing 2 mg Nicotine (from Nicotine Polacrilex)

Large scale preparation of 2 mg nicotine (from nicotine polacrilex) tablets were prepared using a process similar to that described herein above in Example 1. In order to achieve large scale production, the formulation ingredient amounts were adjusted to accommodate the inclusion of microcrystalline cellulose, colloidal silicon dioxide, and flavoring agents.

The formulation prepared is set forth in the following table:

TABLE 5
200 mg Tablets containing Nicotine Polacrilex (Large Scale)

| Ingredient: | mg/tablet | | % w/w |
|---|---|---|---|
| Nicotine polacrilex (15%) | 13.33 | | 6.67 |
| Mannitol (spray-dried) | 52.42 | | 26.21 |
| Sodium bicarbonate | 42.00 | | 21.00 |
| Citric acid | 30.00 | | 15.00 |
| Silicified microcrystalline cellulose | 25.00 | | 12.50 |
| Sodium carbonate | 20.00 | | 10.00 |
| Sodium starch glycolate | 10.00 | | 5.00 |
| Magnesium stearate | 4.00 | | 2.00 |
| Natural and artificial mint flavor | 2.50 | | 1.25 |
| Colloidal silicon dioxide | 0.75 | | 0.38 |
| Total: | 200.00 | mg | 100.0% |

### Example 6

### Comparative In Vivo Pharmacokinetic Data in Humans

The PK (pharmacokinetic) parameters associated with five formulations of solid nicotine dosage forms were tested in humans. The five formulations included the formulations of Table 6 (Treatment A), Table 7 (Treatment B), Table 8 (Treatment C), Table 9 (Treatment D), and the commercial product, COMMIT® (Treatment E; described in Example 4).

TABLE 6
Treatment A - 2 mg, 5/16″ Effervescent Nicotine Polacrilex Tablet

| Ingredient: | mg/tablet | |
|---|---|---|
| Nicotine polacrilex (15%) | 13.33 | |
| Mannitol (spray-dried) | 50.42 | |
| Sodium bicarbonate | 42.00 | |
| Citric acid | 30.00 | |
| Silicified microcrystalline cellulose | 25.00 | |
| Sodium carbonate | 20.00 | |
| Sodium starch glycolate | 10.00 | |
| Magnesium stearate | 4.00 | |
| Natural and artificial mint flavor (SN027513) | 2.50 | |
| Micronized sucralose | 2.00 | |
| Colloidal silicon dioxide | 0.75 | |
| Total: | 200.00 | mg |

TABLE 7
Treatment B - 2 mg, 5/16″ Non-Effervescent Nicotine Polacrilex Tablet

| Ingredient: | mg/tablet | |
|---|---|---|
| Nicotine polacrilex (15%) | 13.33 | |
| Mannitol (spray-dried) | 142.42 | |
| Silicified microcrystalline cellulose | 25.00 | |
| Sodium starch glycolate | 10.00 | |
| Magnesium stearate | 4.00 | |
| Natural and artificial mint flavor (SN027513) | 2.50 | |
| Micronized sucralose | 2.00 | |
| Colloidal silicon dioxide | 0.75 | |
| Total: | 200.00 | mg |

TABLE 8
Treatment C - 2 mg, 5/16″ Effervescent Nicotine Bitartrate Tablet

| Ingredient: | mg/tablet | |
|---|---|---|
| Nicotine bitartrate (35%) | 6.14 | |
| Mannitol (spray-dried) | 57.61 | |
| Sodium bicarbonate | 42.00 | |
| Citric acid | 30.00 | |
| Silicified microcrystalline cellulose | 25.00 | |
| Sodium carbonate | 20.00 | |
| Sodium starch glycolate | 10.00 | |
| Magnesium stearate | 4.00 | |
| Natural and artificial mint flavor (SN027513) | 2.50 | |
| Micronized sucralose | 2.00 | |
| Colloidal silicon dioxide | 0.75 | |
| Total: | 200.00 | mg |

TABLE 9
Treatment D - 2 mg, 5/16″ Non-Effervescent Nicotine Bitartrate Tablet

| Ingredient: | mg/tablet | |
|---|---|---|
| Nicotine bitartrate (35%) | 6.14 | |
| Mannitol (spray-dried) | 149.61 | |
| Silicified microcrystalline cellulose | 25.00 | |
| Sodium starch glycolate | 10.00 | |
| Magnesium stearate | 4.00 | |
| Natural and artificial mint flavor (SN027513) | 2.50 | |
| Micronized sucralose | 2.00 | |
| Colloidal silicon dioxide | 0.75 | |
| Total: | 200.00 | mg |

Twenty-four normal, healthy subjects were enrolled in the study.

All subjects were included in the safety analysis. One subject was did not complete the study and was excluded from PK summary statistics and statistical comparisons. Additionally, subjects with predose nicotine concentrations greater than 5% of Cₘₐₓ were excluded from PK summary statistics and statistical comparisons.

Subjects were dosed with each of the five treatments in one of five treatment sequences (ABCDE, BCDEA, CDEAB, DEABC, and EABCD). Subjects were assigned a unique subject number (1-24) in the order in which they were dosed. The dose period schedule is shown in Table 10.

Subjects reported to the clinic at least 48 hours prior to the first dosing (Period 1, including Day −2 and Day −1) in order to undergo a 48-hour nicotine washout, and remained confined through completion of the 24-hour post-dose procedures for Period 5. A washout of 24 hours separated dosing of each study period. A single dose was administered at Hour 0 on Day 1 of each period by the clinic staff. The meals for Day −2 and −1 of Period 1 were standard clinic meals consisting of breakfast, lunch, dinner, and an evening snack, served at appropriate times. The meals following dosing for each period were identical and consisted of breakfast, served at least 2 hours following dosing, lunch approximately 5 hours following dosing, dinner approximately 9 hours following dosing, and an evening snack following the 12-hour postdose blood draw at a time that met the required 10-hour overnight fast prior to the next dose. Subjects were required to fast for a minimum of 10 hours overnight prior to each study drug administration.

Subjects washed and dried their hands prior to dosing. Subjects were instructed to swallow saliva before dosing. The unit dose container for each subject was opened by clinic staff immediately prior to administration; however, the subjects placed each treatment (tablet or lozenge) in their own mouth.

Subjects were instructed that they would take the tablet and place it between the upper gum and cheek, above a molar tooth. The tablet was not placed above the incisor tooth. The subjects were instructed to let the tablet melt and to not chew it or move it around in the mouth. The treatments could possibly have caused increased salivation. Subjects were instructed to retain the saliva in the mouth for as long as possible, without swallowing, to allow drug absorption from the oral mucosa; clinic staff explained to subjects that this was facilitated by maintaining the head tilted slightly forward. Subjects were instructed to refrain from talking except to answer questions posed by clinic staff.

TABLE 10
Dose Period Schedule

| | Period 1 | | | | | | | | | | | | | | | | | | | | | |
|---|---|---|---|---|---|---|---|---|---|---|---|---|---|---|---|---|---|---|---|---|---|---|
| | only | | Periods 1, 2, 3, 4, and 5 | | | | | | | | | | | | | | | | | | | |
| | Study Day | | | | | | | | | | | | | | | | | | | | | |
| | −2 | −1 | | | | | | | | | | | | | | | | | | | | 2 |
| | Study Time | | | | | | | | | | | | | | | | | | | | | |
| | | | | | 10 | 15 | 20 | 25 | 30 | 40 | 50 | 1.0 | 1.25 | 1.5 | 2 | 3 | 4 | 6 | 8 | 10 | 12 | 24 |
| | −48 h | −24 h | 0 h | 5 min | min | min | min | min | min | min | min | h | h | h | h | h | h | h | h | h | h | h |
| Event | | | | | | | | | | | | | | | | | | | | | | |
| Oral Exam | X | | X | | | | | | | | | | | | | | X | | | | | |
| Complete Vital | X | | | | | | | | | | | | | | | | | | | | | |
| Signs | | | | | | | | | | | | | | | | | | | | | | |
| Adverse Events | Monitored for and documented throughout the study | | | | | | | | | | | | | | | | | | | | | |
| PD Vitals | | | X | | | | | | | | | | | | | | | | | | | |
| PK Blood Draw | | | X | X | X | X | X | X | X | X | X | X | X | X | X | X | X | X | X | X | X | X1 |
| Dose | | | X | | | | | | | | | | | | | | | | | | | |

| | |
|---|---|
| Confinement | Confined from check-in of Period 1 (at least 48 hours predose) through completion of Period 5 (24 hours postdose) |

1 - Period 5 only
PD Vitals - Pharmacodynamic vital measurements - blood pressure and pulse
PK Blood Draw - Blood draw for pharmacokinetic analysis - 4 mL blood sample for plasma nicotine analysis collected in a plastic EDTA evacuated tube prior to dose and at the indicated times

At each dosing, clinic staff instructed the subject as to which side of the mouth they would be using. The left and right sides of the mouth were to be used alternately for each CIMA tablet (e.g., for a subject with the sequence of B, C, D, E, A, they used B/right; C/left; D/right; and A/left) unless qualified medical staff observed a medical reason for not alternating to the other side.

The subject placed the tablet as instructed. Upon placement, the position was verified as correct by clinic staff and the side of the mouth used was documented. Subjects were instructed to notify the clinic staff when they felt the tablet had completely dissolved and to then continue to refrain from talking until clinic staff verified disintegration of the tablet. The clinic staff then checked the appropriate area of the oral cavity to verify the disappearance of the tablet and document the time so that dwell times could be calculated.

Dwell time is defined as lapsed time between the insertion of the tablet or lozenge and disappearance of same from the site of application. Complete disintegration did not mean that all tablet-related material was dissolved. Some residue may have remained but the tablet had disintegrated and there was no remaining “core” of the tablet.

For dose E, subjects were instructed to place the lozenge in their mouth per the COMMIT® package instructions. In addition, subjects were asked to alert the clinic staff upon complete elimination of the lozenge but otherwise refrain from talking until clinic staff verified disintegration of the lozenge unit. The clinic staff then checked the oral cavity to verify the disappearance of the lozenge-related material and verify the time so that dwell times could be calculated. If complete disintegration had not occurred, the subject continued to wait and notify the clinic staff again upon disintegration. At 20 minutes post-dose, a clinic staff member visually inspected the mouth of those subjects who had not experienced complete disintegration of the lozenge. Any remaining portion of the lozenge was allowed to disintegrate on its own with clinic staff visually inspecting the mouth every 5 minutes (or upon subjects' notification of disintegration). The time of “complete disintegration” was noted on the appropriate source document.

Four milliliters of blood were drawn prior to dose and at each time indicated in Table 10 for measuring plasma nicotine. Plasma concentrations of nicotine were determined at MDS Pharma Services in Lincoln, Nebr. using high performance liquid chromatography-tandem mass spectrometry (HPLC/MS/MS) methods validated with respect to accuracy, precision, linearity, sensitivity, and specificity. The lower limit of quantitation (LLOQ) for nicotine was 0.200 ng/mL.

Results

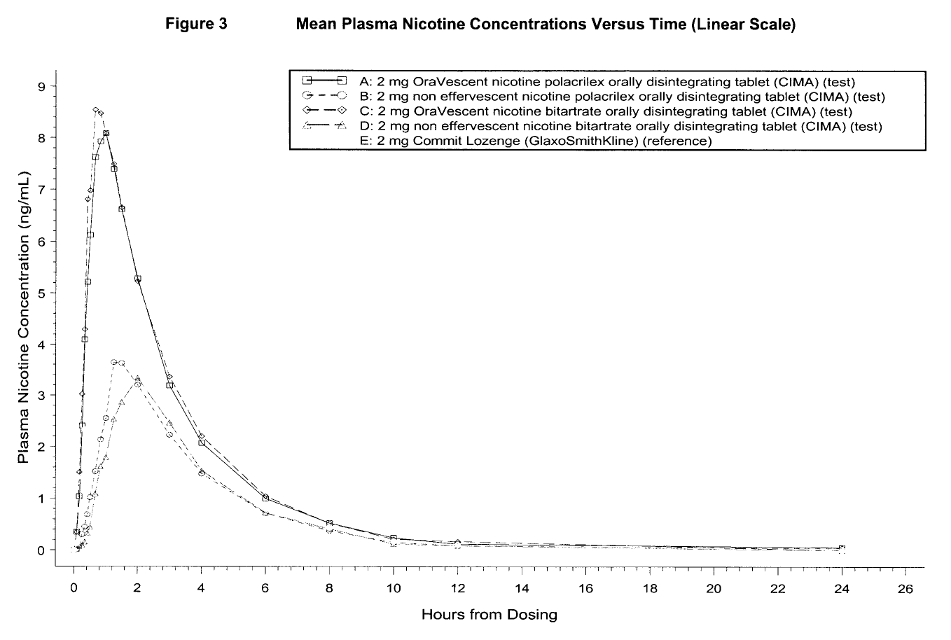

The mean peak nicotine exposure (Cₘₐₓ) was 79% to 96% higher following administration of the 2 mg effervescent nicotine polacrilex (Treatment A) or the 2 mg effervescent nicotine bitartrate (Treatment C) compared to following the administration of 2 mg COMMIT® Lozenge (Treatment E). Additionally, the mean peak nicotine exposure (Cₘₐₓ) was 22% to 28% lower following administration of the 2 mg non effervescent nicotine polacrilex (Treatment B) or the 2 mg non effervescent nicotine bitartrate (Treatment D) compared to following the administration of 2 mg Commit® Lozenge (Treatment E). See FIG. 3 and Table 11.

TABLE 11
Pharmacokinetic Parameters for Nicotine Treatments

| | Treatment A | Treatment B | Treatment C | Treatment D | Treatment E |
|---|---|---|---|---|---|
| PK Parameter | Mean ± SD | Mean ± SD | Mean ± SD | Mean ± SD | Mean ± SD |
| Cₘₐₓ (ng/mL) | 9.242 ± 2.530 | 4.048 ± 1.297 | 10.107 ± 2.608 | 3.723 ± 1.206 | 5.159 ± 1.360 |

### Nicotine Dosage Form on Holder

In an alternative embodiment to the tablet dosage forms described above is a larger lozenge-type dosage form can be prepared in which the dosage form provided herein can be modified into a lozenge affixed or removably attached to a holder or stick. Such dosage form on holder embodiments can be prepared as described in co-pending U.S. Patent Application Ser. Nos. 60/872,177 and 60/872,125, both of which were filed on Dec. 1, 2006—the texts of which are incorporated herein by reference.

According to this particular embodiment, the behavioral aspects of nicotine addiction and smoking are addressed by the presence of the holder or stick, which permits the user to mimic the presence of a cigarette. In this embodiment, the oral dosage form as described herein is coupled to one end of the holder, such that the user can maintain the dosage form adjacent to the mucosal tissue and ensure continual positioning adjacent the mucosal tissue by manipulating the holder by hand.

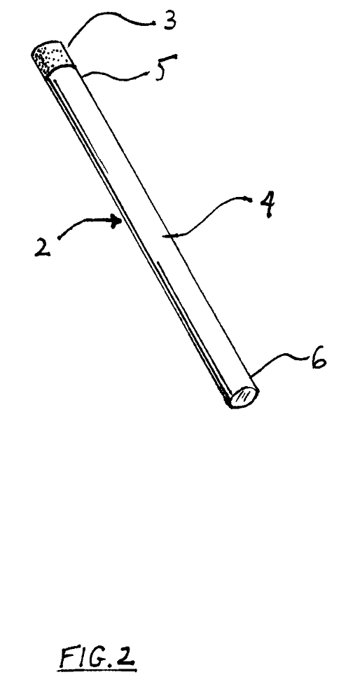

Referring now to FIG. 2, a dosage form on holder system 2 is shown according to one embodiment. The system 2 can comprise a holder portion 4 and dosage form 3 coupled to the holder portion 4. The holder portion 4 can be dimensioned in a variety of configurations and sizes. In one embodiment and as shown, the holder portion 4 (and dosage form 3) can be constructed according to the typical dimensions of a cigarette. The holder portion 4 can contain two ends—an oral end 5 for placement within the recipient's mouth, and a grasping end 6. The holder portion 4 can be constructed using a variety of materials. Suitable materials include those materials that can afford flexible semi-rigid or rigid structure to facilitate grasping and manipulation of the system by the hand, and such materials can include a variety of plastics and paper materials. The dosage form 3 can be attached to the holder portion 4 a variety of attachment means (not specifically shown), including non-toxic adhesives or glues, coupling structures such as pegs, as an exterior coating, and the like.

As the dosage forms described herein can be either fixed to a holder or constructed for reversible detachment from a holder, the user can be afforded the option of converting a lollipop-type nicotine delivery system into a free-standing discrete lozenge or dosage form per se according to the user's preferences.

For the particular embodiment wherein the dosage form and holder are reversibly separable to one another, the dosage form contains a reversible coupling structure. The reversible coupling structure can be constructed as: 1) a dosage form structure, e.g., a recess or cavity, which can receive or accommodate an end of the holder; 2) a structure located on the end of a holder, e.g., a friction enhancing texture, which can removably retain the holder in or on the dosage form; or a combination of both such structures. The holder can further include indicia. Examples of indicia include brandnames, logos, symbols, dosage information, instructions, colors, and the like. Indicia can be applied using various techniques and equipment, such as molding, impressing or embossing techniques, adhesive labeling, and the like, readily available to those skilled in the art.

The holder can further be constructed on the grasping end to include friction-enhancing features, such as tackifiers or pebbling textures. Alternatively and/or in addition to such features, the grasping end can contain finger-specific structures such as tabs and curves.

The present disclosure includes reference to various and specific embodiments and techniques. It will be understood by one skilled in the art, however, that reasonable modifications and variations can be made from the embodiments and techniques without significant departure from either the spirit or scope of the disclosure.

What is claimed is:

1. A solid oral transmucosal dosage form comprising:
a) nicotine or a nicotine derivative or a pharmaceutically acceptable salts thereof,
b) an effervescent system; and
c) a pH adjusting substance;
the dosage form being formulated for resident placement within a recipient's oral cavity for transmucosal delivery of the nicotine or nicotine derivative across the recipient's oral mucosal tissue.

2. The dosage form of claim 1, wherein the pH adjusting substance is present in a amount sufficient to provide a localized pH in the range of about 4 to about 10.

3. The dosage form of claim 2, wherein the effervescent system comprises an acid and an effervescent base.

4. The dosage form of claim 3, wherein the effervescent base and the pH adjusting substance are the same compound.

5. The dosage form of claim 3, wherein the effervescent base is an alkali metal carbonate salt, an alkali metal bicarbonate salt, an alkaline earth carbonate, or an alkaline earth bicarbonate.

6. The dosage form of claim 3, wherein the acid is citric acid.

7. The dosage form of claim 3, wherein the effervescent system is present in amount of about 5% to about 95% by weight of the total dosage form.

8. The dosage form of claim 7, wherein the effervescent system comprises citric acid and an alkali or alkaline earth bicarbonate salt.

9. The dosage form of claim 7, wherein the effervescent system comprises citric acid or tartaric acid and an alkali or alkaline earth bicarbonate salt and the pH adjusting agent is an alkali or alkaline earth carbonate salt.

10. The dosage form of claim 1, wherein the effervescent system comprises citric acid or tartaric acid and an alkali or alkaline earth bicarbonate salt; and the pH adjusting agent is an alkali or alkaline earth carbonate salt; wherein the effervescent system is present in amount of about 25% to about 50% by weight of the total dosage form.

11. The dosage form according to claim 10, wherein the dosage form composition is in the form of a 200 mg total weight oral buccal transmucosal tablet containing nicotine derivative from about 0.5 mg to about 4.0 mg, the tablet having a diameter of about 5/16 inch.

12. The dosage form according to claim 10, wherein the nicotine derivative is selected from the group consisting of nicotine polacrilex and nicotine bitartrate.

13. The dosage form according to claim 1, wherein the dosage form upon administration achieves a Cₘₐₓ to dose ratio ranging from about 3 to about 12 picogram/mL/microgram.

14. The dosage form according to claim 1, wherein the dosage form upon administration achieves a Cₘₐₓ to dose ratio ranging from about 3.7 to about 6.3 picogram/mL/microgram.

15. A solid oral transmucosal dosage form comprising the following ingredients:
a) nicotine or nicotine derivative or a pharmaceutically acceptable salts thereof, and
b) an effervescent system consisting essentially of an acid and an alkali metal or alkaline metal bicarbonate or carbonate salt;
the dosage form being formulated for resident placement within a recipient's oral cavity for transmucosal delivery of the nicotine or nicotine derivative across the recipient's oral mucosal tissue; wherein the bicarbonate or carbonate salt is present in stoichiometric excess relative to the acid and the stoichiometric excess is sufficient to provide a localized pH in the range of about 4 to about 10.

16. The dosage form of claim 15, wherein the effervescent system is present in amount of about 25% to about 50% by weight of the total dosage form.

17. The dosage form according to claim 15, wherein the dosage form upon administration achieves a Cₘₐₓ to dose ratio ranging from about 3 to about 6.3 picogram/mL/microgram.

18. A method of treating nicotine addiction in a recipient desiring such treatment, the method comprising:
a) providing to the recipient a solid oral transmucosal dosage form comprising:
i) nicotine or nicotine derivative or a pharmaceutically acceptable salts thereof,
ii) an effervescent system; and
iii) a pH adjusting substance;
the dosage form being formulated for resident placement within a recipient's oral cavity for transmucosal delivery of the nicotine or nicotine derivative across the recipient's oral mucosal tissue;
b) positioning the dosage form within the recipient's oral cavity adjacent to oral mucosal tissue; and
c) permitting the dosage form to reside in such position for a period of time sufficient to permit the nicotine or nicotine derivative to transport across the oral mucosal tissue;
wherein step c) provides a Cₘₐₓ to dose ratio in a range of from about 3 to about 12 picogram/mL/microgram.

19. An oral transmucosal nicotine delivery system, the system comprising:
a) a solid oral transmucosal dosage form comprising a composition comprising the following ingredients:
i) nicotine or nicotine derivative or a pharmaceutically acceptable salt thereof,
ii) an effervescent system; and
iii) a pH adjusting substance;
the dosage form being formulated for placement within a recipient's oral cavity for transmucosal delivery of the nicotine or nicotine derivative across the recipient's oral mucosal tissue; in combination with
b) a holder;
the dosage form being coupled to an end of the holder.

20. The system of claim 19, wherein the holder is a hand-held stick.

21. The system of claim 19, the delivery system provides a localized pH in the range of about 7 to about 9.

22. The system of claim 19, wherein the effervescent system is present in an amount of about 25% to about 50% by weight of the total dosage form.

23. The system according to claim 19, wherein the holder and dosage form are constructed for reversible coupling to one another.

24. A method of nicotine substitution comprising:
a) providing a dosage form to a recipient desiring the substitution, the dosage form being a solid oral transmucosal dosage form comprising:
i) nicotine or nicotine derivative or a pharmaceutically acceptable salt thereof,
ii) an effervescent system; and
iii) a pH adjusting substance;
the dosage form being formulated for placement within a recipient's oral cavity for transmucosal delivery of the nicotine or nicotine derivative across the recipient's oral mucosal tissue;
b) placing the dosage form within the recipient's oral cavity adjacent to recipient's mucosal tissue; and
c) permitting the dosage form to reside adjacent the mucosal tissue for a period of time sufficient to deliver nicotine across the mucosal tissue.

25. The method of claim 19, the delivery system provides a localized pH in the range of about 4 to about 10.

26. The method of claim 19, wherein the effervescent system is present in an amount of about 25% to about 50% by weight of the total dosage form.
